(19) United States
(12) Patent Application Publication
(10) Pub. No.: US 2017/0049700 A1
(43) Pub. Date: Feb. 23, 2017

(54) SALICYLIC ACID COMPOSITION

(71) Applicant: Exeltis USA Dermatology, Inc., Florham Park, NJ (US)

(72) Inventors: Mats Silvander, Uppsala (SE); Jeffrey S. Day, Newtown, PA (US); Linda M. Mahoney, Phoenixville, PA (US); Christopher Nelson Hensby, Fort Worth, TX (US)

(21) Appl. No.: 15/340,881

(22) Filed: Nov. 1, 2016

Related U.S. Application Data
(63) Continuation of application No. 12/579,713, filed on Oct. 15, 2009, now abandoned.
(60) Provisional application No. 61/105,557, filed on Oct. 15, 2008.

(51) Int. Cl. A61K 9/12; A61K 47/02; A61K 47/10; A61K 47/26; A61K 31/60; A61K 9/00

(52) U.S. Cl. CPC A61K 9/12; A61K 9/124; A61K 31/60; A61K 9/0014; A61K 47/10; A61K 47/26; A61K 47/02

(57) ABSTRACT

Provided, among other things, is a delivery module for water-based salicylic acid composition comprising: an aerosol delivery system; within the aerosol delivery system, the salicylic acid composition comprising 0.5% or more salicylic acid by weight, lipophilic component(s), and a frothing agent, the salicylic acid composition having a viscosity low enough to support aerosol delivery, and the salicylic acid composition effective to form a foam upon propellant-driven aerosol delivery; and within the aerosol delivery system, a propellant, wherein the salicylic acid composition is non-irritating and has a non-watery feel.

This application claims the priority of Ser. No. 61/105,557, filed 15 Oct. 2008.

The present invention relates to a composition of salicylic acid, which can be used to treat acne, psoriasis, calluses, corns, keratosis pilaris, warts, dandruff, and the like.

The dermatological utility of salicylic acid has been attributed to its causing skin cells to slough off Commercial available topical compositions are often 17% (w/w). Salicylic acid has been reported to be antiseptic and antifungal. Salicylic acid can also be used to treat dermatitis, such as Lichen simplex.

Dermatological compositions of salicylic acid have been formulated in oily bases (lotions) and gels. Oil-based formulations provide a protective layer and localize the salicylic acid on the skin. These oil-based also facilitate formulating salicylic acid at useful concentrations and at the relatively low pH values that facilitate the dermatological actions of salicylic acid. Gel-based products facilitate formulation with a relatively large aqueous phase.

For foam-forming compositions based on emulsions, the compounding issues for salicylic acid are significant. Prior art foam formulations and formulating methods are susceptible to having the salicylic acid form non-uniformities such as lumps. These formulations and formulating methods tend to use alcohols and are also susceptible to insufficient foaming, and insufficient retention of water after application of the foam to a patient. When applied to a patient, the foams tend to melt or breakdown, with the alcohol evaporating.

A format that has been used for making dermatological foams is that of a urea product on the market. That product is believed to rely heavily on oils, such as Shea butter and sunflower oil, though it is said to have some amount of stearic acid. Given the amount of oils, this format may be usable for salicylic acid. When used to deliver urea, the format leaves a wet, watery layer at the site of application.

The present invention initially addressed many of these problems with a foam. It has now believed that many of these advantages are obtained with emulsions formulated as creams, gels, lotions, milks, and the like.

### SUMMARY OF THE INVENTION

Provided, in one embodiment, is a delivery module for water-based salicylic acid composition comprising: an aerosol delivery system; within the aerosol delivery system, the salicylic acid composition comprising 0.5% or more salicylic acid by weight, lipophilic component(s), and a frothing agent, the salicylic acid composition having a viscosity low enough to support aerosol delivery, and the salicylic acid composition effective to form a foam upon propellant-driven aerosol delivery; and within the aerosol delivery system, a propellant, wherein the salicylic acid composition is non-irritating and has a non-watery feel. The composition in the system can contain, for example, by weight: salicylic acid 0.5-10%; optionally fatty acid(s) and/or analogous alkyl amine(s) and/or polyalkyleneglycol-fatty acid ester(s) 0.005-10% ; hydrophilic polymer(s) 0.05-5%; and frothing agent(s) 3-11%. In certain embodiments, the polyalkyleneglycol-fatty acid ester component is the predominant component among the fatty acid, analogous alkyl amine and polyalkyleneglycol-fatty acid ester components.

Also provided, in one embodiment, is a salicylic acid composition comprising: A. salicylic acid 0.5-10%; B. optionally fatty acid(s) and/or analogous alkyl amine(s) and/or polyalkyleneglycol-fatty acid ester(s) 0.005-10%; C. hydrophilic polymer(s) 0.05-5%; and D. frothing agent(s) 1-11%, wherein the salicylic acid composition is effective to form a foam, and is non-irritating and has a non-watery feel. Additionally provided is a method of formulating the salicylic acid composition comprising adding the salicylic acid in an oil phase to a water solution comprising substantially all of hydrophilic polymer(s), the admixture providing substantially all of the components A through D. This composition also provides a non-irritating and non-watery feel in a bulk form, prior to aerosol delivery, and may in fact be delivered without a propellant, in the non-aerosolized form, or other emulsion forms such as gels, creams, lotions, and the like.

Further provided, in another embodiment, is a method of treating acne, psoriasis, calluses, corns, keratosis pilaris, dermatitis, warts or dandruff comprising applying an aerosol-driven foam to affected skin a foamed, non-greasy, water-based salicylic acid composition comprising: the salicylic acid composition comprising 0.5% or more salicylic acid by weight, lipophilic component(s), and a frothing agent, the salicylic acid composition having a viscosity low enough to support aerosol delivery, and the salicylic acid composition effective to form a foam upon propellant-driven aerosol delivery, wherein the salicylic acid composition is non-irritating and has a non-watery feel.

Also provided, in another embodiment, is a method of treating acne, psoriasis, calluses, corns, keratosis pilaris, dermatitis, warts or dandruff comprising applying to affected skin a non-greasy, water-based salicylic acid composition comprising: the salicylic acid composition comprising 0.5% or more salicylic acid by weight, lipophilic component(s), and a frothing agent, wherein the salicylic acid composition is non-irritating and has a non-watery feel.

### DETAILED DESCRIPTION OF THE INVENTION

In certain embodiments, the formulation of the invention provides a non-irritating foam. Irritation is measured by ISO 10993-10: 2002 Standard, “Biological Evaluation of Medical Devices, Part 10-Tests for Irritation and Sensitization,” pp. 6-10, 21, which testing method is incorporated herein by reference. In particular, for each test site on shaved dorsal skin of an albino rabbit, gauze incorporating 0.5 mL of test material or negative control material is applied. One test and one control site are used on each side of the paravertebral skin. The infused gauzes are covered with tape-backed gauze. The trunk of the rabbit is wrapped in elastic bandage secured by hypoallergenic tape. After a minimum of 24 hours, the coverings are unwrapped. Observations are made at 60 min±2, 24 h±2, 48 h±2 and 72 h±2 post unwrapping. Tissue reactions are rated for gross evidence of erythema and edema.

For a given rabbit, values for each test site and each of the 24 h, 48 h and 72 h measurements are totaled, and divided by six (2 tests sites×3 measurements). Control values were treated in the same way. For all rabbits, these test values were summed, normalized against the summed values for the negative controls, and divided by the number of animals. A negligible, slight, moderate or severe response is categorized based on the Primary Irritation Index:

| Response Category | Comparative Mean Score |
|---|---|
| Negligible | 0 to 0.4 |
| Slight | 0.5 to 1.9 |
| Moderate | 2 to 4.9 |
| Severe | 5 to 8 |

By “non-irritating” it is meant that compositions according to this embodiment of the invention illicit a Negligible Primary Irritation Index.

The non-irritating quality of these embodiments is surprising in view of the surfactants often found in these embodiments. While not being bound by theory, it is believed that water and appropriate selection of relatively mild surfactants, as illustrated herein, may contribute to the non-irritating quality of the foam.

In certain embodiments, the foam of the invention has a “non-greasy feel” when applied. A non-greasy feel is measured in reference to a comparison of the feel of the Example 1 composition (non-greasy standard) of U.S. application Ser. No. 12/016,371, filed Jan. 18, 2008 (US2008/175793), applied to skin at 1 mg/cm², compared to the oil-based product described in the Table at Column 3 of U.S. Pat. No. 5,919,470 (Bradley Pharmaceuticals, Inc., greasy standard), applied in the same amount. Application includes working the foam into the skin. While the feel of compositions of the invention may vary, in making the comparison between the non-greasy standard, the greasy standard, and the prospective non-greasy composition, it will be apparent which category the prospective composition falls within. The non-greasy skin feel may be moist and smooth feeling, but the difference in greasy feel relative to the greasy comparative shall be clear.

In certain embodiments, the foam of the invention has a “non-watery feel” when applied. A non-watery feel is a feel much like that of the Example 1 composition (non-watery standard) of U.S. application Ser. No. 12/016,371, filed Jan. 18, 2008 (US2008/175793), applied to skin at 1 mg/cm². A feel that, in contrast, is substantially more watery, is disqualified.

In certain embodiments, the foam of the invention is a stable foam, meaning that when applied to the skin at one of 1, 2 or 3 mg/cm² and not worked into the skin, the foam remains a stably adherent foam for 30 seconds or more. In some cases, the foam remains a stably adherent foam for 60 seconds or more, 120 seconds or more, 150 seconds or more or 180 seconds or more. While stable, the foam can be worked into the patient's skin.

In certain embodiments, the foam-forming composition of the invention is essentially free of C1 to C6 alcohols. In certain embodiments, the foam-forming composition is essentially free of C1 to C5 alcohols. In certain embodiments, the foam-forming composition is essentially free of C1 to C4 alcohols. By essentially free it is meant that such alcohols may be present in minor amounts, as may be useful for example for compounding, but are not present in an amount that one of skill in the art of pharmaceutical foam formulating would select to stabilize the salicylic acid or the emulsion of a foam-forming composition. In these embodiments, the amount of such alcohols is less than about 8 wt %. In certain embodiments, the amount of such alcohols is than about 5%, or 2%, or 1% (wt/wt).

When worked into the skin, the compositions of the invention can have rapid absorption—contributing to their non-greasy and non-watery feels. The compositions can be easy to spread and are cosmetically elegant.

Salicylic acid can be present in dermatologically effective amount. For example, it can be present in an amount from A or above, from B or below, or from A to B (inclusive, optionally exclusive, of the endpoints), where A is 0.5, 1.0, 1.5, 2.0, 2.5, 3.0, 3.5, 4.0, 4.5, 5.0, 5.5, 6.0, 6.5, 7.0, 7.5, 8.0, 8.5, 9.0 or 9.5% wt; and B is 2.5, 3.0, 3.5, 4.0, 4.5, 5.0, 5.5, 6.0, 6.5, 7.0, 7.5, 8.0, 8.5, 9.0, 9.5, 10.0%, 15% or 20% wt. (All ranges in this specification are inclusive, and optionally exclusive, of the endpoints.)

The composition can contain lipophilic components (inclusive of acid forms of salicylic acid) that are believed to help distribute salicylic acid (inclusive of its salts) on and into the skin. A major portion of such lipophilic components can be amphiphates in amounts effective to stabilize the lipophilic components in solution and/or emulsified. Example amphiphates are fatty acids, which can be substantially or essentially ionized, wherein the salt is soluble in the aqueous solution of the salicylic acid composition. Other example amphiphates are polyalkyleneglycol-fatty acid esters. Further examples are alkyl amines with one alkyl per amine having a size distribution analogous to that of an appropriate fatty acid composition. Further examples are nonionic detergents.

The lipophilic components are, in certain embodiments, non-greasy, meaning that in the aggregate of the formulation, as formulated in the foam-forming composition, they are non-greasy.

The fatty acid can, for example, be of any composition found in a natural source, including hydrolysis of esterified fatty acids. Or, the fatty acid component can be hydrogenated to remove substantially all or a portion of any unsaturation. In certain embodiments, the fatty acid component or the alkyl moiety of the alkyl amine component is selected such that 50 mole % or more is C12 or higher, or C14, or C16 or higher. In certain embodiments, the fatty acid component or the alkyl moiety of the alkyl amine component is selected such that 50 mole % or more is C22 or lower, or C20 or lower, or C18 or lower. In certain embodiments, 75 mole % or more of the fatty acid component is from C12 or C14 or C16 to C22 or C20 or C18. In certain embodiments, 80 mole % or more, 85 mole % or more, 90 mole % or more, 95 mole % or more, 97 mole % or more, 98 mole % or more, or 99 mole % or more, meets one of the size parameters of this paragraph. In certain embodiments, the fatty acyl component of polyalkyleneglycol-fatty acid esters falls in one of the above ranges.

For carboxylic acid containing lipophilic components, useful salts include the alkali metal salts such as sodium or potassium salts; ammonium salts; salts formed with suitable organic bases, such as amine salts (such as triethyl amine, triethanol amine, or the like) and quaternary ammonium salts; or the like. Bivalent or trivalent salts can be used where they do not adversely affect solubility. For amine-containing lipophilic components, useful salts include maleates, fumarates, lactates, oxalates, methanesulfonates, ethanesulfonates, benzenesulfonates, tartrates, citrates, halides (e.g., hydrochlorides, hydrobromides), sulfates, phosphates, nitrates, and the like. As needed, the lipophilic components are provided such that a sufficient amount of constituent ionizable molecules are in ionized (salt) form to provide solubility. Such ionized forms can be prepared by adding a titrant. Recitations of compositions described by their formation by titration include the equivalent compositions formed by pre-formed salts or otherwise.

The alkyl component of polyalkyleneglycol-fatty acid ester is generally C2-C5, but predominantly C2. For example, ethyleneglycol can comprise 51% or more, 55% or more, 60% or more, 65% or more, 70% or more, 75% or more, 80% or more, 85% or more, 90% or more, 95% or more, 100% of the glycol units (molar basis). The number of glycol repeat units is generally a number from C or above, from D or below, or from C to D, where C is 10, 15, 20, 25, 30 or 35, and D is 60, 55, 50 or 45.

In certain embodiments, where present, the fatty acid, analogous alkyl amine, or polyalkyleneglycol-fatty acid ester components together (to the extent present) comprise an amount of E or more, F or less, of from E to F of the foam-forming composition, where E is 0.005, 0.008, 0.01, 0.05, 0.1,0.5, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6 wt %, and F is 0.02, 0.05, 0.1, 0.5, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4,4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8. 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9 or 10 wt %. Unless otherwise specified, the composition percentages for the foam-forming compositions are exclusive of propellant, such as propane or butane or the like.

In certain embodiments, the polyalkyleneglycol-fatty acid ester comprises an amount of E or more, F or less, of from E to F of the foam-forming composition. In certain embodiments, the amount of polyalkyleneglycol-fatty acid ester, among amphiphates in the foam-forming composition, is an amount of G or more, H or less, or from G to H of the amphiphates, where G is 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 wt %, and H is 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 71, 72, 73, 74, 75, 76, 77, 78, 79 or 80 wt %. In certain embodiments, the polyalkyleneglycol-fatty acid ester comprises a predominant portion of the fatty acid, analogous alkyl amine, and polyalkyleneglycol-fatty acid ester components.

An emollient, if present, can be a silicone oil such as polydimethylsiloxane (i.e., dimethicone), petrolatum, or the like. In certain embodiments, the emollient(s) are an amount I or more, J or less, or I to J of the foam-forming composition, where I is 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9 or 4 wt %, and J is 0.6, 0.7, 0.8, 0.9, 1, 1.1, 1.2, 1.3, 1.4, 1.5, 1.6, 1.7, 1.8, 1.9,2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 wt %. In certain embodiments, as among emollients and amphiphates in the foam-forming composition, the amount of emollient is an amount K or more, L or less, or K to L of the emollients and amphiphates, where K is 5, 6, 7, 8, 9, 10, 11, 12, 13, 14, 15, 16, 17, 18, 19 or 20 wt %, and L is 6, 7, 8, 9, 10, 11, 12, 13, 14, 15, 16, 17, 18, 19, 20, 21, 22, 23, 24 or 25 wt %.

The amphiphates will typically include frothing agents, which for non-foam embodiments can be termed. Frothing agents can be non-ionic detergents, such as polyoxyethylene sorbitan fatty acid esters (such as Tween 80 (polyoxyethylene (20) sorbitan monolaurate), Polysorbate 20 (polyoxyethylene (20) sorbitan monooleate)), sorbitol fatty acid esters, octyly glucosides, PEGylated lipids and the like. In certain embodiments, the frothing agent(s) comprise an amount of M or more, N or less, of from M to N of the foam-forming composition, where M is 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6 wt %, and N is 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9 or 11 wt %. For certain non-foam embodiments, the surfactant(s) comprise an amount of M′ or more, N′ or less, of from M′ to N′ of the foam-forming composition, where M′ is 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4,4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6 wt %, and N′ is 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9 or 11 wt %. The frothing agent(s) can comprise detergents with 2 or more, 3 or more, 4 or more, 5 or more fold difference in CMC. The frothing agents can, for example, have a CMC at 21° C. of 2×10⁻⁶ M to 10⁻⁴ M. In certain embodiments, where there are two or more frothing agents, the predominant (by wt) frothing agent can have the lower CMC vs the next most predominant frothing agent.

Hydrophilic polymer(s) can be present. These can be any non-toxic water soluble polymer(s) that (in the aggregate) stabilize foam and contribute to film formation on the skin. Examples include polyvinyl pyrrolidone, polyethylene glycol, starch, water-soluble derivatives of starch, cellulose, methyl cellulose, hydroxymethylcellulose, other water-soluble derivatives of cellulose, carbamers, or the like. For polyvinyl pyrrolidone, for example, useful average molecular weights include from 8,000 to 63,000, such as about 38,000. For all polymers used in the composition, the size can be sufficient to limit penetration of the horny layer of the skin, if skin penetration is an issue for the given polymer. In certain embodiments, hydrophilic polymer(s) are an amount O or more, P or less, or O to P of the foam-forming composition, where 0 is 0.05, 0.1, 0.2, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9 or 4 wt %, and P is 0.6, 0.7, 0.8, 0.9, 1, 1.1, 1.2, 1.3, 1.4, 1.5, 1.6, 1.7,1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 wt %.

The composition can also contain a humectant, such as glycerol, propylene glycol, other polyols, polydextrose, lactic acid, or the like. In certain embodiments, humectant(s) are an amount M or more, N or less, or M to N of the foam-forming composition.

The wound-treating composition will typically contain a preservative or preservative system. Examples include Phenonip™ XB (a mixture of preservatives, believed to include phenoxyethanol, methylparaben, ethylparaben, butylparaben, propylparaben and isobutylparaben; from Clariant UK Ltd., Leeds, UK), or a less complex preservative, such as one or two of methylparaben, ethylparaben, butylparaben, propylparaben and isobutylparaben.

The foam-forming composition will typically contain titrating agents such as triethylamine, NaOH, citrate, and the like. The amount is typically selected to provide a dermatologically acceptable pH, such as pH 4-8.

The salicylic acid compositions can be formulated as creams, lotions, gels, milks, foam-formers, and the like. Where creams or lotions are desired, these consistencies can be obtained by selection of hydrophilic polymers, and the amounts thereof. For example, these can include polymers that have a greater effect on increasing viscosity, in appropriate amounts. Such polymers can include, for example, appropriate carbamers, methy cellulose, hydroxyl alkyl cellulose, gum arabica, and the like. Addition of suitable hydrophilic co-polymer permits the formation of different emulsion dosage forms that retain the same safety and efficacy properties as the foam but do not require the use of gaseous propellants for their delivery to the treatment area. In some cases, the amount of surfactant is reduced.

Suitable propellants include, for example, propane, butane, isobutene, other hydrocarbons, hydrofluorocarbons, chlorofluorocarbons (Cl/F/(H)/C), and the like. Dispensing systems include those available from Deutsche Prazision, Lindal Group (Schonberg, Germany), Coster (Milano, Italy) and SeaquistPerfect Dispensing (Cary, Ill.).

The invention further provides methods of formulating the foam-forming composition comprising adding the salicylic acid in an oil phase to a water solution comprising substantially all of hydrophilic polymer(s), the admixture providing substantially all of the components that are a, salicylic acid, b, fatty acid(s) and/or analogous alkyl amine(s) and/or polyalkyleneglycol-fatty acid ester(s), c, hydrophilic polymer(s), and d, frothing agent(s). In certain embodiments, more oil-compatible humectants are proved in the oil phase, and relatively more hydrophilic humectants arc added in the water solution.

To formulate 100 g, one can formulate all or a selection of the formulations defined by the combinations of the following options:

| | Component | Amt. Options (g) |
|---|---|---|
| C1 | Povidone | 1.3, 1.5, 1.7 |
| C2 | Polyoxyethylene 40 stearate | 3.0, 4.0, 5.0 |
| C3 | Methyl cellulose (1500 cp) | 0.5, 1.0 |
| C4 | Stearic acid | 0.013, 0.016 |
| C5 | Sodium citrate | 2.0, 3.0 |
| C6 | Glycerol | 1.0, 2.0 |
| C7 | Triethanol amine | 2.5, 3.0 |
| C8 | Preservative | 0.5 |
| B | NaOH | As needed |
| A5 | Salicylic acid | 6.0 |
| A2 | Dimethicon | 2.0, 2.5 |
| A1 | Propylene glycol | 4.0, 5.0, 6.0 |
| A3 | Tween 80 | 4.0, 5.0 |
| A4 | Polysorbat 20 | 2.0, 3.0 |
| | Water | Quantity Sufficient |

The above can be formulated in 3 phases: mixing the A components; mixing B in a minor amount of the water; mixing the C components in the bulk of the water; adding the mixed A components to the mixed C components; and adding the mixed B components. The A components can be added to A1 stepwise in the order A2 to A5. The C components can be added to water stepwise in the order C1 to A8, with the water heated to promote mixing and solubilization. The mixed A components can be added in parts to the mixed C components, such as after the mixed A components have cooled, but still have an elevated temperature (over r.t.). The mixed B components are added (to A+B) after further cooling. The formulations can be tested for foam forming, foam stability, non-wet feel, irritation, non-greasy feel, and the like.

### Effective Amount

To treat the indications of the invention, an effective amount of a salicylic acid will be recognized by clinicians but includes an amount effective to treat, reduce, alleviate, ameliorate, eliminate or prevent one or more symptoms of the disease sought to be treated or the condition sought to be avoided or treated, or to otherwise produce a clinically recognizable favorable change in the pathology of the disease or condition. In effective amount can be a dermatological treatment effective concentration of salicylic acid.

### Misc. Embodiments

The invention further encompasses, among other things, the following numbered embodiments:

Embodiment 1. A delivery module for water-based salicylic acid composition comprising: an aerosol delivery system; within the aerosol delivery system, the salicylic acid composition comprising 0.5% or more salicylic acid by weight, lipophilic component(s), and a frothing agent, the salicylic acid composition having a viscosity low enough to support aerosol delivery, and the salicylic acid composition effective to form a foam upon propellant-driven aerosol delivery; and within the aerosol delivery system, a propellant, wherein the salicylic acid composition is non-irritating and has a non-watery feel.

Embodiment 2. The delivery module of one of embodiments 1 or 3-7, wherein the salicylic acid composition comprises, by weight: salicylic acid 0.5-10%; optionally fatty acid(s) and/or analogous alkyl amine(s) and/or polyalkyleneglycol-fatty acid ester(s) 0.005-10%; hydrophilic polymer(s) 0.05-5%; and frothing agent(s) 3-11%.

Embodiment 3. The delivery module of one of embodiments 1-2 or 4-7, wherein the polyalkyleneglycol-fatty acid ester component is the predominant component among the fatty acid, analogous alkyl amine and polyalkyleneglycol-fatty acid ester components.

Embodiment 4. The delivery module of one of embodiments 1-3 or 5-7, wherein the salicylic acid composition provides a stable foam.

Embodiment 5. The delivery module of one of embodiments 1-4 or 6-7, wherein the salicylic acid composition is essentially free of Cl-C6 alcohols.

Embodiment 6. The delivery module of one of embodiments 1-5 or 7, wherein the salicylic acid composition provides a non-greasy feel.

Embodiment 7. The delivery module of one of embodiments 1-6 , wherein the salicylic acid comprises 2-10%, and hydrophilic polymer(s) comprise 0.5-5%.

Embodiment 8. A salicylic acid composition comprising: A. salicylic acid 0.5-10%; B. optionally fatty acid(s) and/or analogous alkyl amine(s) and/or polyalkyleneglycol-fatty acid ester(s) 0.005-10%; and C. hydrophilic polymer(s) 0.05-5%; D. surfactant(s) 1-11%, wherein the salicylic acid composition is non-irritating and has a non-watery feel.

Embodiment 9. The delivery module of one of embodiments 8 or 10-13, wherein the polyalkyleneglycol-fatty acid ester component is the predominant component among the fatty acid, analogous alkyl amine and polyalkyleneglycol-fatty acid ester components.

Embodiment 10. The delivery module of one of embodiments 8-9 or 11-13, wherein the surfactants comprise 3-11% and the salicylic acid composition provides a stable foam.

Embodiment 11. The salicylic acid composition of one of embodiments 8-10 or 12-13, wherein the salicylic acid composition is essentially free of C1-C6 alcohols.

Embodiment 12. The salicylic acid composition of one of embodiments 8-11 or 13, wherein the salicylic acid composition provides a non-greasy feel.

Embodiment 13. The salicylic acid composition of one of embodiments 8-12, wherein the salicylic acid composition is effective to form a foam, the salicylic acid comprises 2-10%, and hydrophilic polymer(s) comprise 0.5-5%.

Embodiment 14. A method of treating acne, psoriasis, calluses, corns, keratosis pilaris, warts or dandruff comprising applying a non-greasy, water-based salicylic acid composition comprising: the salicylic acid composition comprising 0.5% or more salicylic acid by weight, lipophilic component(s), and a frothing agent, wherein the salicylic acid composition is non-irritating and has a non-watery feel.

Embodiment 15. The method of one of embodiments 14 or 16-21, wherein applied composition comprises fatty acid, analogous alkyl amine or polyalkyleneglycol-fatty acid ester, and wherein the polyalkyleneglycol-fatty acid ester component is the predominant component among the fatty acid, analogous alkyl amine and polyalkyleneglycol-fatty acid ester components

Embodiment 16. The method of one of embodiments 14-15 or 17-21, wherein the salicylic acid composition provides a stable foam.

Embodiment 17. The method of one of embodiments 14-16 or 18-21, wherein the salicylic acid composition is essentially free of C1-C6 alcohols.

Embodiment 18. The method of one of embodiments 14-17 or 19-21, wherein the salicylic acid composition provides a non-greasy feel.

Embodiment 19. The method of one of embodiments 14-18 or 20-21, wherein the applied composition comprises: salicylic acid 0.5-10%; optionally fatty acid(s) and/or analogous alkyl amine(s) and/or polyalkyleneglycol-fatty acid ester(s) 0.005-10%; hydrophilic polymer(s) 0.05-5%; and frothing agent(s) 3-11%.

Embodiment 20. The method of one of embodiments 14-19 or 21, comprising applying an aerosol-driven foam to affected skin a foamed, non-greasy, water-based salicylic acid composition comprising: the salicylic acid composition comprising 2% or more salicylic acid by weight, lipophilic component(s), and a frothing agent, the salicylic acid composition having a viscosity low enough to support aerosol delivery, and the salicylic acid composition effective to form a foam upon propellant-driven aerosol delivery, wherein the salicylic acid composition is non-irritating and has a non-watery feel.

Embodiment 21. The method of one of embodiments 14-21, wherein the applied composition comprises: salicylic acid 0.5-10%; optionally fatty acid(s) and/or analogous alkyl amine(s) and/or polyalkyleneglycol-fatty acid ester(s) 0.005-10%; hydrophilic polymer(s) 0.5-5%; and frothing agent(s) 3-11%.

Embodiment 22. A method of formulating the composition of one of embodiments 8-13 comprising adding the salicylic acid in an oil phase to a water solution comprising substantially all of hydrophilic polymer(s), the admixture providing substantially all of the components A through D.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred systems and methods may be used and that it is intended that the invention may be practiced otherwise than as specifically described herein. Accordingly, this invention includes all modifications encompassed within the spirit and scope of the invention as defined by the claims that follow.

1-25. (canceled)

26. A method of manufacturing an aerosol delivery system for dispensing a stably adherent foam composition, the method comprising:
mixing a composition salicylic acid, one or more frothing agents, one or more humectants, one or more surfactants, one or more emollients, water, and one or more preservatives;
providing a propellant system comprising butane and propane; and
providing the composition and the propellant system in the aerosol delivery system.

27. The method of claim 26, wherein the mixing comprises mixing a first set of ingredients comprising salicylic acid, propylene glycol, polysorbate 20 to produce a first mixture.

28. The method of claim 27, wherein the mixing comprises mixing a second set of ingredients comprising sodium hydroxide in water to produce a second mixture.

29. The method of claim 28, wherein the mixing comprises mixing a third set of ingredients comprising glycerin and water to produce a third mixture.

30. The method of claim 29, wherein the water in the third set of ingredients is heated to promote mixing and solubilization.

31. The method of claim 29, wherein the first mixture is added to the third mixture to produce a combined mixture.

32. The method of claim 31, wherein the first mixture is added in parts to the third mixture.

33. The method of claim 31, wherein the second mixture is added to the combined mixture.

34. The method of claim 31, wherein the second mixture is added to the combined mixture.

35. The method of claim 26, wherein the one or more humectants selected from the group consisting of glycerin and propylene glycol.

36. The method of claim 26, wherein the one or more preservatives selected from the group consisting of methylparaben, propylparaben, butylparaben, isobutylparaben, and phenoxyethanol.

37. The method of claim 26, wherein the one or more propellants selected from the group consisting of: butane, propane.

38. The method of claim 26, wherein the one or more titrants selected from the group consisting of: triethanolamine, sodium hydroxide.

39. The method of claim 26, wherein the one or more frothing agents selected from the group consisting of polyoxyethylene sorbitan fatty acid esters, polyoxyethylene (20) sorbitan monolaurate, polysorbate 20, sorbital fatty acid esters, octyly glucosides, and PEGylated lipids.

40. The method of claim 26, wherein the composition does not contain petrolatum.

41. An aerosol delivery system for dispensing a stably adherent foam composition produced from the method of claim 26.

42. A method for treating one or more skin diseases in a subject comprising:
applying the stably adherent foam composition disposed from the aerosol delivery system of claim 26 onto a skin of a subject that is afflicted with one or more skin diseases;
wherein the applying elicits a negligible primary irritation index in the skin of the subject as measured by ISO 10993-10; and
wherein the applying treats, reduces, alleviates, ameliorates, eliminates, or prevents one or more symptoms of the one or more skin diseases.

43. The method of claim 42, wherein the one or more skin diseases are selected from the group consisting of: acne, psoriasis, calluses, corns, keratosis, pilaris, dermatitis, warts, and dandruff

44. The method of claim 43, wherein the applying comprises applying at least 1 mg/cm² of the stably adherent foam composition onto the skin of the subject and allowing the stably adherent foam to remain on the skin for at least 30 seconds.

45. The method of claim 44, further comprising working the stably adherent foam onto the skin of the subject after the applying.
